(19) United States
(12) Patent Application Publication
(10) Pub. No.: US 2024/0026263 A1
(43) Pub. Date: Jan. 25, 2024

(54) MODELING, MONITORING AND MANUFACTURING OF MULTI-ORGAN SYSTEMS FOR HUMAN PHYSIOLOGY AND PATHOLOGY

(71) Applicant: John Collins, Irvine, CA (US)

(72) Inventor: John Collins, Irvine, CA (US)

(21) Appl. No.: 18/477,549

(22) Filed: Sep. 29, 2023

Related U.S. Application Data
(63) Continuation-in-part of application No. 16/948,734, filed on Sep. 30, 2020, now Pat. No. 11,807,842.

(51) Int. Cl. C12M 3/00; C12M 3/06; C12M 1/00; C12M 1/34; C12N 5/071

(52) U.S. Cl. CPC C12M 21/08; C12M 23/16; C12M 23/40; C12M 41/46; C12N 5/0697

(57) ABSTRACT

Gravity-driven microfluidic system provides unidirectional physiological flow in cells, organs, multi-organs and organoids culture. Such gravity-driven flow is integrated in multi-organ system on a plate or multi-organ system on a chip to provide recirculations that simulates blood flow in humans. In addition, mechanical actuations on the organs provide true human on a chip or true human on a plate platform. Stretching of the organ substrate using gas at controlled pressure profile provides muscular stimulation and culturing the stretched organ at air/media or gas/liquid interface is useful for organ simulations.

### CROSS-REFERENCE TO RELATED APPLICATIONS

This patent application is a continuation-in-part of U.S. application Ser. No. 16/948,734 filed on Sep. 30, 2020. The above application is incorporated by reference herein.

### STATEMENT REGARDING FEDERALLY-SPONSORED RESEARCH AND DEVELOPMENT

This invention was made with Government support under award numbers 1R43ES032357, 1R44GM139413 and 1R43AG073040 awarded by the National Institute of Health (NIH). The Government has certain rights in this invention.

### FIELD OF THE INVENTION

The present invention generally relates to medical devices and methods, and more particularly to microfluidic devices and methods for modeling, manufacturing and monitoring organ and human functions for understanding disease mechanism, drug discovery and toxicology in organ plates.

### BACKGROUND OF THE INVENTION

Microfluidic system provides assays that are useful for various physiological and pathophysiological applications such as Alzheimer's Disease, diabetes, toxicology, infectious disease, isogenic organs modeling, cancer models. There is a need in the art for Gravity-driven microfluidic systems that provide unidirectional physiological flow in cells, organs, multi-organs and organoids culture.

### BRIEF INTRODUCTION

Gravity-driven microfluidic system provides unidirectional physiological flow in cells, organs, multi-organs and organoids culture. Such gravity-driven flow is integrated in multi-organ system on a plate or multi-organ system on a chip to provide recirculations that simulates blood flow in humans. In addition, mechanical actuations on the organs provide true human on a chip or true human on a plate platform. Stretching of the organ substrate using gas at controlled pressure profile provides muscular stimulation and culturing the stretched organ at air/media or gas/liquid interface is useful for lung organ. Modeling multi-organ on a chip or multi-organ on a plate will provide shear flow rate to organs, mechanical stress, barrier functions and endothelial/epithelial co-culture across organs. Monitoring field potential signals from brain, cardiac and muscular organs may be used to analyze the functional activity of organs. Further, transepithelial endothelial electrical resistance helps in the monitoring of barrier functions. 3-D confocal imaging of organs cultured on chips or plates will provide monitoring organ functions in morphology or coupled with other immunochemical assays. Time domain measurement of effluents from the organs at various compartments within physiological barriers can help in the proteins, enzymes or analytes measurements. End-point measurement of organ tissues at various compartments are useful for studying immunohistochemical gene expression or protein expression or real time quantitative polymerase chain reaction gene expression. Such assays are useful for various physiological and pathophysiological applications such as Alzheimer's Disease, diabetes, toxicology, infectious disease, isogenic organs modeling, cancer models.

### SUMMARY

The present invention is directed to systems, devices, chips, plates, and methods for cells, organs, organoids, multiple organs or human culture and manufacture in a 2-D or 3-D arrays using a multi-organ plate equipped with gravity-driven liquid flow, pneumatic pressure and/or electro-active polymer-based mechanical stretching of membrane, non-invasive instantaneous measurement techniques, effluent sampling-based assays and endpoint immunochemical and genomic assays from the organ plate, as shown in and/or described in connection with at least one of the figures, and as set forth more completely in the claims.

In accordance with an aspect of the present invention, there are systems, devices, and methods for performing gravity-driven recirculations in 2-D or 3-D for cells or organs or organoids or multiple organs in one or a plurality of adjacent channels such as endothelium channel, epithelium channel and media channel.

In accordance with yet another aspect of the present invention, there are systems, devices, and methods for connecting multiple central nervous systems and peripheral nervous system or brain and other body organs through blood-brain-barrier (BBB) organ by gravity-driven flow across multiple fluidic loops.

In accordance with yet another aspect of the present invention, there are systems, devices, and methods for connecting multiple channels adjacent to the main channel in the fluidic loop.

In accordance with yet another aspect of the present invention, there are systems, devices, and methods for connecting multiple loops adjacent to middle channels.

In accordance with yet another aspect of the present invention, there are systems, devices, and methods for connecting multiple adjacent channels to coculture multiple cell types or gel or media to middle channels or channel loops.

In accordance with yet another aspect of the present invention, there are systems, devices, and methods for forming vertical walls in the channel by tilting the organ plate at an angle.

In accordance with another aspect of the present invention, there are systems, devices, and methods for loading a fluid within an adjacent channel using Laplace flow-stop side or bridge channels.

In accordance with yet another aspect of the present invention, there are systems, devices, and methods for different heights for the side or bridge channel to enable the flow-stop.

In accordance with yet another aspect of the present invention, there are systems, devices, and methods for different above-mentioned heights for the side or bridge channel to enable the flow-stop one fluid while providing flowthrough for another fluid.

In accordance with yet another aspect of the present invention, there are systems, devices, and methods for flow-stop using micropillars in a linear array or in a loop to stop one fluid entering another region or another fluid.

In accordance with yet another aspect of the present invention, there are systems, devices, and methods the above mentioned micropillars are nested in adjacent channels to stop multiple fluids or regions.

In accordance with yet another aspect of the present invention, there are systems, devices, and methods for different diameters for fluidic channel inlets or outlets.

In accordance with yet another aspect of the present invention, there are systems, devices, and methods for measuring cellular or organ parameters such as transepithelial-endothelial electrical resistance (TEER) or field potential (FP) or impedance using integrated electrodes at the bottom of the organ plate.

In accordance with yet another aspect of the present invention, there are systems, devices, and methods for measuring cellular or organ parameters such as TEER or FP or impedance using pin electrodes from the lid of the organ plate.

In accordance with yet another aspect of the present invention, there are systems, devices, and methods for connecting multiple fluidic loops of different organs to a main fluidic loop through barrier junctions such as blood brain barrier, cerebral fluid brain barrier, air-blood barrier, kidney fluid-blood barrier, hormone-blood barrier, retinal-blood barrier.

In accordance with yet another aspect of the present invention, there are systems, devices, and methods for flowing different fluids such as gas, liquid or different composition of fluids in multiple fluidic loops of different organs.

In accordance with yet another aspect of the present invention, there are systems, devices, and methods for multiple recirculation units by gravitational flow loops within organs and flow-jump from one organ to another organ through fluidic lid.

In accordance with yet another aspect of the present invention, there are systems, devices, and methods for flow-jump from one organ to one or more organs through fluidic lid as a loop across one or more organs.

In accordance with yet another aspect of the present invention, there are systems, devices, and methods for flow-jump from one organ or more organs through fluidic lid as a one-way flow.

In accordance with yet another aspect of the present invention, there are systems, devices, and methods for flow-jump from one organ or more organs through fluidic lid to a fresh media reservoir or waste media collection reservoir.

In accordance with yet another aspect of the present invention, there are systems, devices, and methods to accommodate transwell inserts in wells or channels to provide barrier functions to the organs.

In accordance with another aspect of the present invention, there are systems, devices, and methods for performing tight junction by loading extracellular matrix gels in one channel and loading to cells in adjacent channels to form vertical walls.

In accordance with yet another aspect of the present invention, there are systems, devices, and methods involving multiple valves, pressure sensors and reservoirs to provide pneumatic positive and negative pressure profiles to organs cultured on membranes.

In accordance with yet another aspect of the present invention, there are systems, devices, and methods to load cells or organs through a port and sealing using silicone-based stoppers as a lid to organ plate and performing mechanical stimulation on the membrane using pneumatic positive and negative pressure control.

In accordance with yet another aspect of the present invention, there are systems, devices, and methods to perform linear or multi-dimensional mechanical stretches on muscular organs supported by a membrane using pneumatic positive and negative pressure control.

In accordance with yet another aspect of the present invention, there are systems, devices, and methods for the above-mentioned stretching using electro-active polymer-based activation.

In accordance with yet another aspect of the present invention, there are systems, devices, and methods for the above mentioned electro-active polymer-based activation where the electroactive polymer form stretches the substrate of seeded organs or cells in a concentric fashion.

In accordance with yet another aspect of the present invention, there are systems, devices, and methods for culturing certain organs such as lungs and skin at air/water interface and communicating to cells or media in adjacent layers through pores in the supporting membrane.

In accordance with yet another aspect of the present invention, there are systems, devices, and methods for pressuring certain membranes holding organs or cells from the top or bottom using gas pressure.

In accordance with yet another aspect of the present invention, there are systems, devices, and methods for culturing certain organs on membranes interfaced to gas on one side and liquid on the other side.

In accordance with yet another aspect of the present invention, there are systems, devices, and methods for transporting gas or liquid from top layer to the bottom layer to flow continuously in a loop.

In accordance with yet another aspect of the present invention, there are systems, devices, and methods for transporting gas or liquid from one loop or another loop through channels in a microfluidic lid.

In accordance with yet another aspect of the present invention, there are systems, devices, and methods for blocking the above-mentioned pores for any applied pneumatic pressure using endothelial cells or other cells forming tight junction.

In accordance with yet another aspect of the present invention, there are systems, devices, and methods for performing organ or cell culture with recirculation and stretching using closed systems.

In accordance with yet another aspect of the present invention, there are systems, devices, and methods for performing organ or cell culture with recirculation using gravity-driven flow or pneumatic driven flow or a combination of flows.

In accordance with yet another aspect of the present invention, there are systems, devices, and methods for stretching the membrane after blocking using pneumatic control of gases.

In accordance with yet another aspect of the present invention, there are systems, devices, and methods for loading fluid on a channel by vertically holding the chip or plate through bottom ports so that any air bubbles will be escaped through the top outlet using gravity.

In accordance with yet another aspect of the present invention, there are systems, devices, and methods for culturing cells or organs on the bottom surface of the membrane by tilting the chip or plate after loading the sample and culturing for an extended period so that the cells or organs are attached to the surface.

In accordance with yet another aspect of the present invention, there are systems, devices, and methods for loading multiple cells in the cells by mixing with gel solution or activator solution so that the cell density the organ plate is a factor for deciding the volume to prepare the cultured cells.

In accordance with yet another aspect of the present invention, there are systems, devices, and methods for different layers of the organ plate compressed in a bracket to hold the fluidics tight within the channel.

In accordance with yet another aspect of the present invention, there are systems, devices, and methods for integrating disposable and sterilizable non-disposable plates to provide a closed environment for organ plates.

In accordance with yet another aspect of the present invention, there are systems, devices, and methods for stacking one or multiple organ plates in multiple trays and performing recirculation within all the organ plates.

In accordance with yet another aspect of the present invention, there are systems, devices, and methods for stacking multiple organ plates in multiple tray-towers to access individual organ plates and performing recirculation within all the organ plates.

In accordance with yet another aspect of the present invention, there are systems, devices, and methods to pick up multiple organ plates from the above-mentioned tray-towers and to sequentially bring them to imaging systems and to media exchange through multiple robotic arms.

In accordance with yet another aspect of the present invention, there are systems, devices, and methods to pick up multiple organ plates during tilting-based recirculation from the above-mentioned tray-towers so that the robotics picks up a plate from the tilted position.

In accordance with yet another aspect of the present invention, there are systems, devices, and methods to remember the tilted positions from tray-towers and to keep track of the positions for picking up one or more organ plates for one or more machines for multiple organ manipulation or multiple organ monitoring.

In accordance with yet another aspect of the present invention, there are systems, devices, and methods for measuring FP signals from brain, heart or muscle organs.

In accordance with yet another aspect of the present invention, there are systems, devices, and methods for measuring TEER from lung, kidney, skin and BBB organs.

In accordance with yet another aspect of the present invention, there are systems, devices, and methods for the measurement of field potential signals from the electrodes on the bottom substrate using spring loaded or edge connectors in printed circuit boards (PCB).

In accordance with yet another aspect of the present invention, there are systems, devices, and methods to connect electrodes by projecting the bottom substrate from the skirt of the organ plate.

In accordance with yet another aspect of the present invention, there are systems, devices, and methods for imaging and performing gravity-driven recirculation continuously or successively during organ culture.

In accordance with yet another aspect of the present invention, there are systems, devices, and methods for markings on organ plates as reference points to locate different organs during imaging.

In accordance with yet another aspect of the present invention, there are systems, devices, and methods for in situ measuring or monitoring oxygen, chemiluminescence, fluorescence, pH and multiple gasses dissolved in the media using electrical or optical probes.

In accordance with yet another aspect of the present invention, there are systems, devices, and methods for measuring or monitoring oxygen, chemiluminescence, fluorescence, pH and multiple gasses dissolved in the media using a multi-modal plate reader with sparse-matrix scan of the organ plate.

In accordance with yet another aspect of the present invention, there are systems, devices, and methods for imaging and image processing with artificial intelligence for analysis of linear, circular, rectangular or irregular objects within the images of organs to characterize biomarkers for physiology or pathology.

In accordance with yet another aspect of the present invention, there are systems, devices, and methods for transporting effluence from the multiple compartments of the organ plate automatically to a well plate spatially and temporally.

In accordance with yet another aspect of the present invention, there are systems, devices, and methods for quantifying different analytes in the effluent from organ plate using multiple methods such as mass spectroscopy/liquid chromatography, immunoassay, spectroscopy and proteomics.

In accordance with yet another aspect of the present invention, there are systems, devices, and methods for performing permeation in the organs within multiple compartments and to perform successive chemical treatment to perform imaging such as immunohistochemistry.

In accordance with yet another aspect of the present invention, there are systems, devices, and methods for transporting fluids from one part of the organ to another during recirculation for live organ culture or mixing in post processing through a microfluidic lid.

In accordance with yet another aspect of the present invention, there are systems, devices, and methods for blocking certain compartments in the flow channel by introducing certain fluids that solidify to close the channel.

In accordance with yet another aspect of the present invention, there are systems, devices, and methods for treating blocked compartments with multiple chemistries for different assays.

In accordance with yet another aspect of the present invention, there are systems, devices, and methods for removing the bottom substrate of the organ plate to retrieve the tissue for proteomics or genomics study.

In accordance with yet another aspect of the present invention, there are systems, devices, and methods for removing the bottom substrate using heat or chemical treatment or mechanical piercing.

In accordance with yet another aspect of the present invention, there are systems, devices, and methods for delivering and removing media using a set of multichannel peristaltic pumps.

Further aspects, elements and details of the present invention are described in the detailed description and examples set forth here below.

### BRIEF DESCRIPTION OF THE DRAWINGS

The embodiments set forth in the drawings are illustrative and exemplary in nature and not intended to limit the subject matter designed by the claims. The following detailed description of the illustrative embodiments may be understood when read in conjunction with the following drawings, where like structures are indicated with like reference numerals in which:

FIG. 1A presents an exemplary diagram of a single unit organ plate with inner and outer loops with multiple organs in the fluidic paths.

FIG. 1B presents an exemplary diagram of a single unit organ plate with multiple adjacent channels to the loops.

FIG. 1C presents an exemplary diagram of multiple channel segments with recirculation loops on either side.

FIG. 1D presents an exemplary diagram of inner channel with adjacent outer channel at different heights.

FIG. 2A presents an exemplary isometric diagram of seeding and culturing cells on a wall of 3-D gel.

FIG. 2B presents an exemplary diagram of co-culture of multiple cell types in adjacent channels.

FIG. 2C presents an exemplary diagram of multiple electrodes for TEER and field potential measurements.

FIG. 2D presents an exemplary diagram of high throughout organ plate with co-culture channels.

FIG. 3A presents an exemplary diagram of micropillars-encapsulated regions for 3-D cell loading regions with two adjacent co-culture regions.

FIG. 3B presents an exemplary diagram of micropillars-encapsulated regions for 3-D cell loading regions with three adjacent co-culture regions.

FIG. 3C presents an exemplary diagram of micropillars-encapsulated regions for 3-D cell loading regions in multiorgan plate for two loops of recirculation.

FIG. 4 presents an exemplary diagram of multiple loops connecting to a main loop for human multi-organ model.

FIG. 5 presents an exemplary diagram of multiple organ systems undergoing intra-organ recirculation and connecting to inter-organs through a microfluidic lid.

FIG. 6A presents an exemplary diagram of a pressure/vacuum pulse generator to mechanically stimulate organs.

FIG. 6B presents an exemplary diagram of an organ plate with an array insert to stimulate organs.

FIG. 6C presents an exemplary diagram of inlet port, outlet port and the membrane connected by fluidic channels.

FIG. 7A presents an exemplary diagram of organ stimulated from bottom gas channel from the supporting membrane.

FIG. 7B presents an exemplary diagram of organ stimulated from top gas channel from the supporting membrane.

FIG. 7C presents an exemplary diagram of multiple layers for the assembling of an organ chip with gas stimulating channel.

FIG. 8A presents an exemplary diagram of electroactive polymer integrated mechanical stimulation chip.

FIG. 8B presents an exemplary diagram of multiple layers for the assembling of electroactive polymer integrated organ chip for mechanical stimulation and imaging.

FIG. 9A presents an exemplary diagram of high throughput organ plate with top gas channel for stimulating organs and bottom fluidic channel for organ culture.

FIG. 9B presents an exemplary diagram of high throughput organ plate with bottom gas channel for stimulating organs and top fluidic channel for organ culture.

FIG. 9C presents an exemplary diagram of multiple layers for assembling high throughput organ plate with top gas channel for stimulating organs and bottom fluidic channel for organ culture.

FIG. 9D presents an exemplary diagram of multiple layers for assembling high throughput organ plate with bottom gas channel for stimulating organs and top fluidic channel for organ culture.

FIG. 10A presents an exemplary diagram of injection-molded multi-organ plate with 24 units.

FIG. 10B presents a flow diagram of multi-step preparation of cells, thrombin and fibrinogen to form 3-d gel to seed the organ plate.

FIG. 10C presents a set of steps for the preparation of cells, thrombin and fibrinogen to form 3-d gel to seed the organ plate.

FIG. 11A presents an exemplary diagram of a mechanical fixture to hold organ plate with top wells and bottom channels.

FIG. 11B presents an exemplary diagram of a closed mechanical fixture to hold organ plate with top wells and bottom channels.

FIG. 11C presents an exemplary diagram of a closed mechanical fixture with lid closing the organ plate for sterility.

FIG. 12A presents an exemplary diagram of multiple organ plates stacked for recirculation.

FIG. 12B presents an exemplary diagram of multiple organ plates stacked for recirculation for automated imaging and media exchange.

FIG. 13A presents an exemplary diagram of organ plate monitoring using chemiluminescence or fluorescence real time assay using optical imaging readout base and light control lid.

FIG. 13B presents an exemplary diagram of organ plate monitoring using transepithelial-endothelial resistance assay using electrode array with PCB and battery on the top.

FIG. 13C presents an exemplary diagram of discretized sparse measurements across the organ plate using a standard 1536 well plate.

FIG. 14A presents an exemplary diagram of organ plate recirculation system.

FIG. 14B presents an exemplary diagram of organ plate recirculation system with microscopic imaging.

FIG. 15A presents an exemplary diagram of timeline for cell-loading in the organ plate and performing imaging assay.

FIG. 15B presents an exemplary diagram of multiple steps of image processing with artificial intelligence algorithms.

FIG. 15C presents an exemplary diagram of workflow in the organ plate for the applications.

FIG. 16A presents an exemplary diagram of automatic effluent sampling from organ plate for multiple times.

FIG. 16B presents an exemplary diagram of media exchange or perfusion in the organ plate.

### DETAILED DESCRIPTION

The following description contains specific information pertaining to implementations in the present application. The drawings in the present application and their accompanying detailed description may be directed to merely exemplary implementations. Unless noted otherwise, like or corresponding elements among the figures may be indicated by like or corresponding reference numerals. Moreover, the drawings and illustrations in the present application are generally not to scale, and are not intended to correspond to actual relative dimensions.

This patent application deals with cell-seeding in an organ plate, and organ cultures using recirculation, media or reagent addition/exchange in the organ plate, media recirculation or media removal in the organ plate, organ in-situ assays or organ growth monitoring, end of experiment in the organ plate or post organ-culture assays, manufacturing of organs in organ plate and applications of organ culture and multi-organ interaction. A multiple unit organ plate system, device, or method may be used instead of a single unit organ plate system, device, or method, as the number of units on an organ plate may be exemplary unless noted otherwise.

In most embodiments, an organ plate provides gravity-driven unidirectional 116A recirculation of media through multiple organs. Multidimensional tilting of organ chip to periodic recirculation with programmable resident times across organs that may be connected in series and parallel. This recirculation fluidic circuit does not require any extra valving circuit for unidirectional flow 116V so that the system may be robust for long term culturing cells. In most embodiments, the organ system may integrate scaled organs developed in static culture protocols from multiple size well plates to enable fluidic shear. Further, Laplace fluidic-stop channels enable layering of extracellular matrix gel and tissues without using artificial membranes, allowing co-culture of endothelium with epithelium and free migration of immune cells through the extracellular matrix gel. In most embodiments, a multiple unit organ plate system, device, or method may be used instead of a single unit organ plate system, device, or method.

### Endothelial/Epithelial Co-Culture—Laplace Stoppers or Micropillars

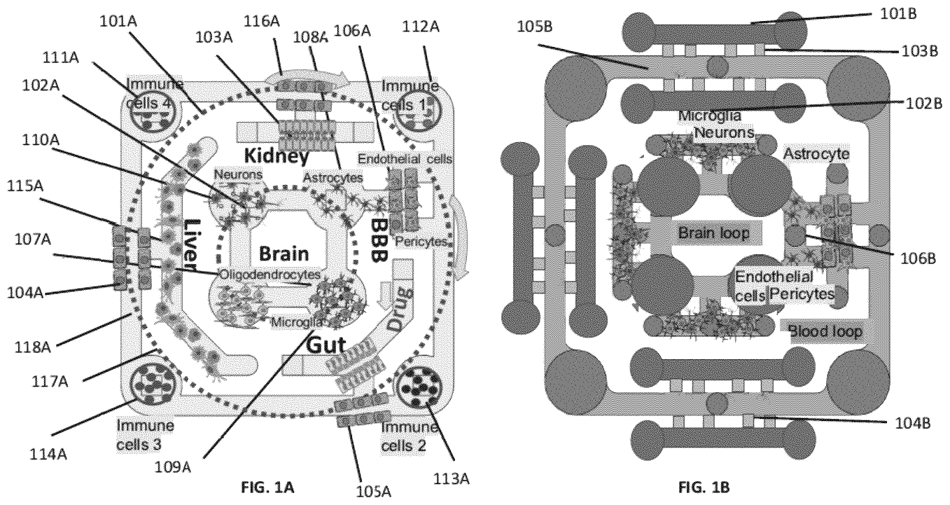

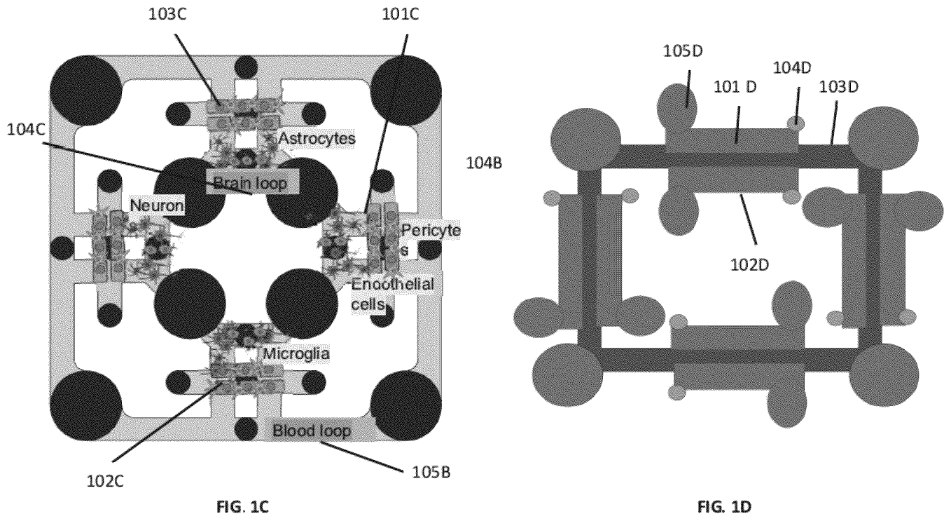

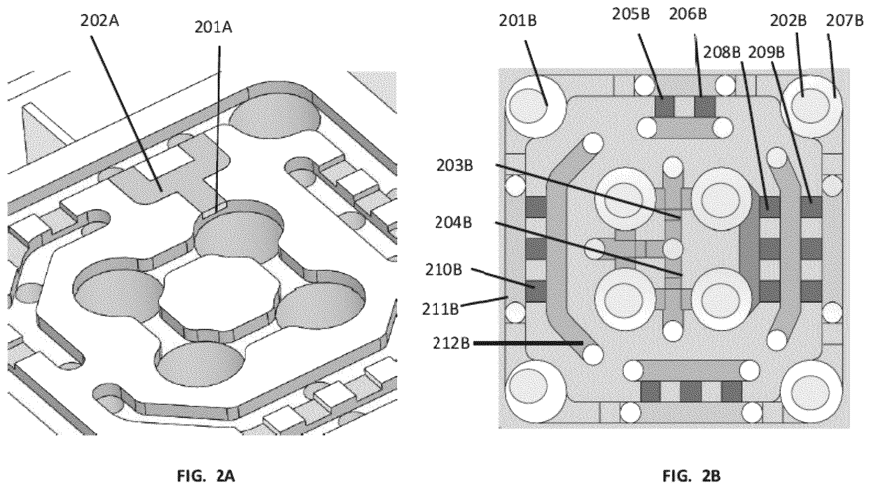

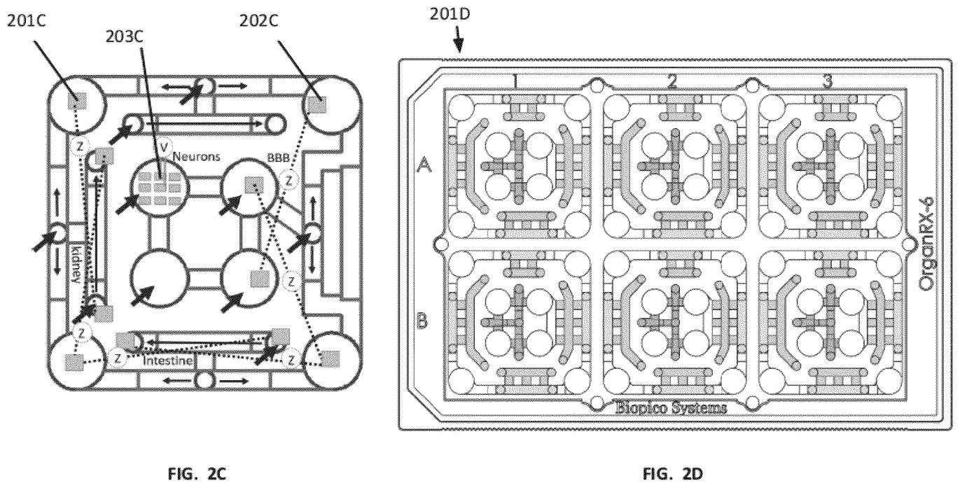

An exemplary design of a single unit organ plate design 10A as shown in FIG. 1A. In most embodiments, a multiple unit organ plate (as shown in FIG. 2D) may be used instead of the single unit organ plate design 10A. In most embodiments, an organ plate 100A contains multiple loops 101A, 102A with multiple organs in series and parallel in the fluidic paths that may be connected to multiple wells. The endothelial and epithelial regions of at least one co-culture of modeled organs 103A, 104A, 105A, 106A may be connected by bridge channels. In some embodiments, the at least one co-culture may be cultured in at least of bridge channels. In some embodiments, co-cultures, cells, and/or cell mixtures, such as multiple brain cells 107A, 108A, 109A, 110A may be cultured in the wells as 2-D or 3-D cultures, either in separate or mixed conditions. In some embodiments, co-cultures, cells, and/or cell mixtures such as immune cells 111A, 112A, 113A, 114A may be cultured on transwell inserts or in gel or floating in media. In most embodiments, gels, liquids, fluids, cells and/or cell mixtures may be delivered into one or more ports of the epithelial region and may stop at the expansion channel area 118A adjacent to the bridges 115A. FIG. 1A also lists, for example, types of cells, organs, and drugs that may be used in the organ plate 100A. Note, the types of cells, organs, and drugs listed the organ plate 100A on FIG. 1A is not exhaustive and may be combined or arranged in any manner as needed by the user.

FIG. 1B shows an organ plate 100B with the multiple adjacent channels 101B, 102B connected through bridge channels 103B, 104B to the main channel 105B for culture of one or more cell types within the organs. Brain loop channel 104C may be connected to the blood loop 105C channel through a single channel 106B or multiple channels 101C, 102C, 103C as shown in FIG. 1C. In most embodiments, the adjacent channels 101D, 102D that may be close to the main channel 103D at a different height to contain each fluid within the channel as shown in FIG. 1D. In some embodiments, the diameter of the fluidic port on one end of the channels to load or vent fluids may be smaller diameter ports 104D while the other end of the channel may be larger 105D. Adjacent channels may also be created by separating them with micropillars 302A, 302B.

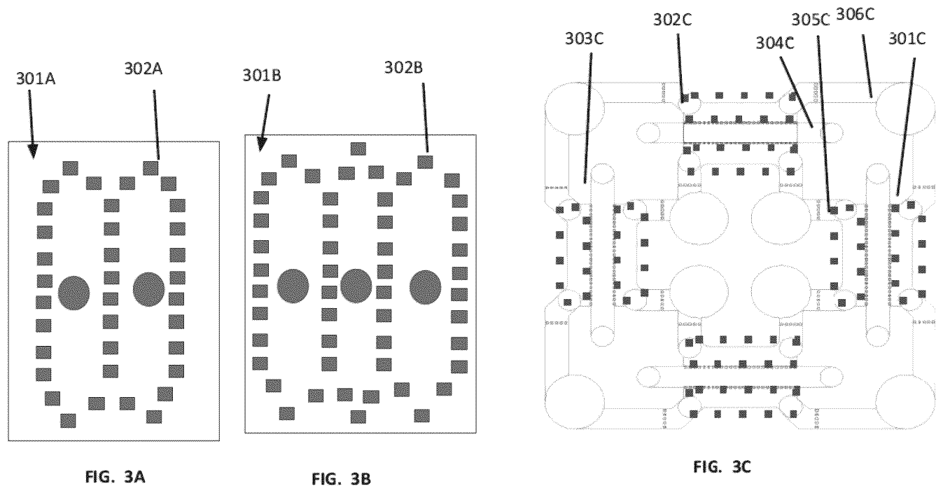

In some embodiments, FIG. 3A shows an example of a two channel device 301A with micropillars 302A and FIG. 3B shows an example of a 3-channel device 301B with micropillars 302B. In another example as seen in FIG. 3C, multiple organs 301C, 302C, 303C may be created across fluidic recirculations 116A in most embodiments. Each organ 301C, 302C, 303C has a middle channel 304C where cells or gel may be loaded. On the side channels 305C, 306C media may be recirculated using gravity driven flow by tilting across the four corners an organ plate 300C.

### Barrier Functions in Organ Plate—Transwell, Vertical Wall, 3-D Gel

In most embodiments, the organ plate cells are seeded on a wall 201A of 3-D gel 202A as shown in FIG. 2A. The cells on the wall may proliferate and form a barrier separating either side of the wall. Further, in some embodiments, barriers formed in transwell inserts 201B, 202B may be integrated in the organ plate by placing the transwell inserts 101B, 202B in the wells 207B as shown in FIG. 2B. In some embodiments, multiple barriers 205B, 206B may be formed across walls of bridge channels 208B, 209B, 210B for different barrier characteristics such as blood-cerebrospinal fluid (CSF) barrier 203B, 204B and endothelial and epithelial barrier 205B, 206B. In some embodiments, the bridge channels 208B, 209B, 210B connecting the endothelial channels 211B and the epithelial channels 212B may be coated with a hydrophobic coating that may prevent and/or avoid fluids crossing the epithelial region into the endothelial region or vice versa. In some embodiments, the hydrophobic coating may be placed on any part of the bridge channels and/or a glass layer of the organ plate. In some embodiments, electrodes such as TEER 201C, 202C, 203C may be integrated in the organ plate to measure impedance, or field potential measurements as shown in FIG. 2C. The electrodes may be integrated into the organ plate and/or placed on a glass layer of the organ plate. In another example, FIG. 2D shows a multiple organ plate 200D with multiple repeating units such as organ plates 200B.

Multi-Organ Fluidic Plate with BBB/Liver/Gut/Kidney/Heart/Skin/Lung/Muscle Organs

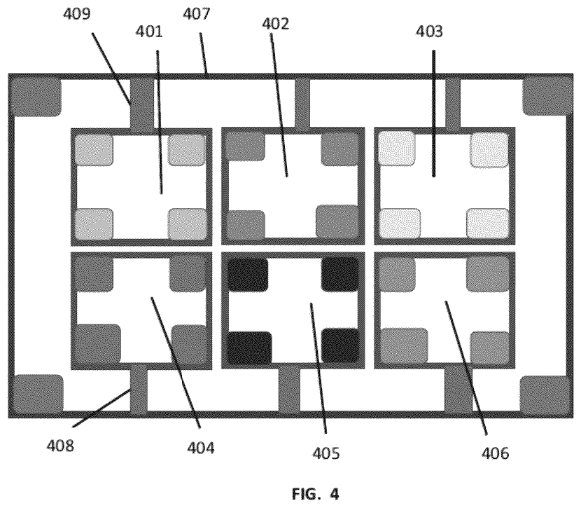

In most embodiments, different organs 401, 402, 403, 404, 405, 406 may be cultured in recirculation loops 101A and/or 117A of an organ plate as shown in FIG. 4. Each co-culture of a modeled organ comprising of at least one blood-brain barrier (BBB), liver, gut, kidney, heart, skin, lung, and muscle, etc., may be connected to a main channel 407 using side channels 408, 409 for organ-organ interactions as in human physiology or pathology. In some embodiments, at least one of the media used in the wells and/or fluid used in the bridge channels may be populated with immune cells so that the immune cells may interact with the co-cultured.

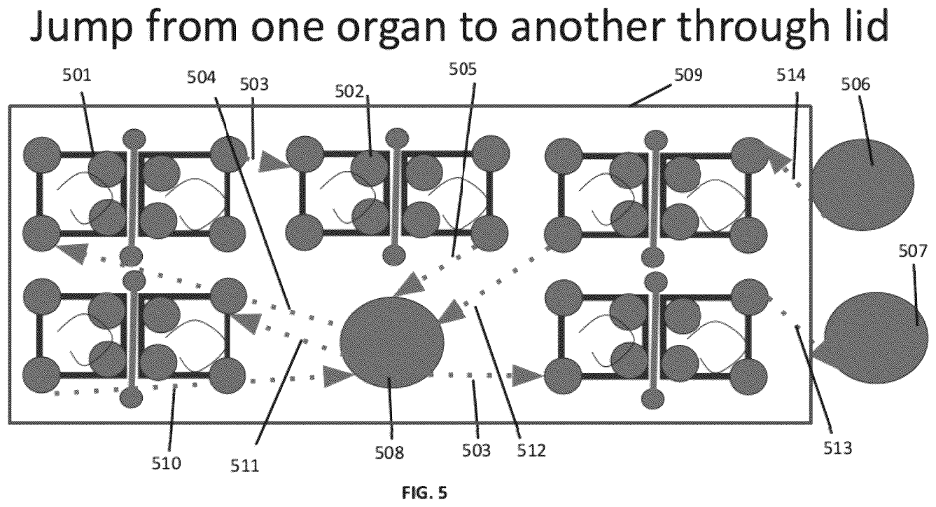

In some embodiments, multiple organs recirculation loops 501, 502 in separate units of the organ plate 500 may be connected to one another, different units, and/or between a set of organ plates 500 for serial or parallel recirculation systems 503, 504, 505 using a fluidic lid 509. The fluidic lid 509 may be configured to connect to each recirculation loop 501, 502 as shown in FIG. 5. In some embodiments, the fluidic lid 509 may be configured to connect to at least one well. In some embodiments, the fluidic lid 509 may be configured to connect the at least one well to a separated well on the organ plate 500. The separate well may be from the same unit, a separate unit on multiple unit organ plate, and/or a second organ plate. In some embodiment, single organ recirculation loops 510, 511 may be connected using the fluidic lid 509. In some embodiments, fresh media 506 may be supplied from an external reservoir 512 through a channel from the fluidic lid 509 and tubing 514 and/or waste media 507 may be directed to a second external reservoir 511 through tubing 513 and channel 503 from the fluidic lid 509. In some embodiments, the media from multiple organs recirculation loops 501, 502 may also be mixed in a mixing reservoir 508 that may be either internal or external to the organ plate 500. In some embodiments, the multiple organ recirculation loops 501, 502 may be mixed in a mixing reservoir 508 that may be connected by a set of organs within the organ plate.

Organ Plate with Fluidics and Stretching

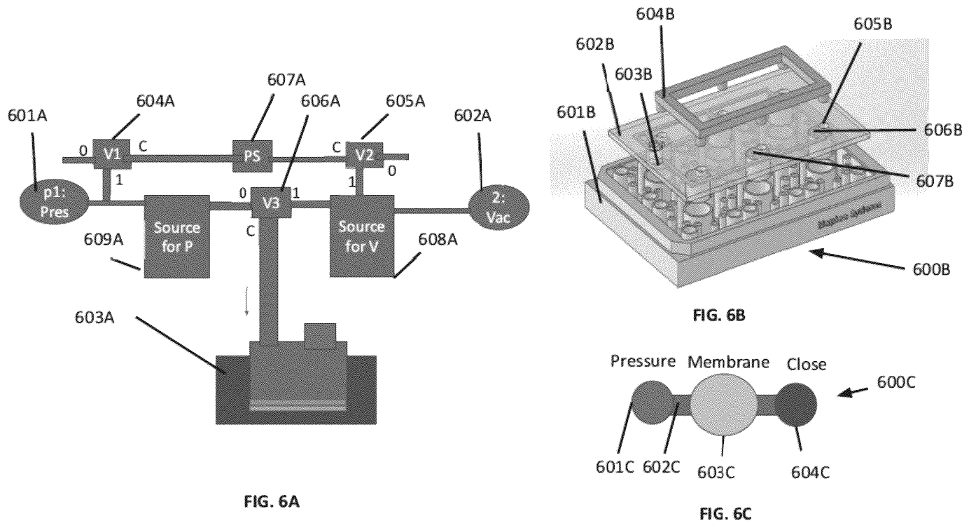

In some embodiments, there may be a need for certain organs such as muscle, heart, lung, etc., that may need mechanical stretching along with fluidic flow. Such organs may be stretched using pneumatic pressure or vacuum source. A pressure or vacuum system 600 may be connected to an organ 603A as shown in FIG. 6A. Pressure pump 601A and vacuum pump 602A may be controlled by a pressure sensor 607A and valves 604A, 605A, 606A to maintain pressure at the sources 608A, 609A. In some embodiments, the pressure and vacuum pulses may be multiplexed to the organ device 603A for stretching the organ. In most embodiments, the stretching of the co-culture mimics the aging, maturing, and or stimulating of the co-culture of the at least one modeled organ.

In another embodiment, the organ plate 601B as shown in FIG. 6B may have a six transwell plate lid 602B where one of the ports 606B may be connected through microfluidic channels to a common luer lock 603B for applying pressure and vacuum pulses to the organ plate 601B. At least one second port 605B may be in each transwell insert 607B may be used for delivering at least one of the media, cells, and gel. In some embodiments, the second port 605B may be closed with a pneumatic seal using a rubber lid 604B. In some embodiments, organ cells may be placed in the middle of the organ plate 600C on a membrane 603C that may be used for stretching as shown in FIG. 6C. Pressure pulses may be applied on one side of the organ plate 600C via a first port 601C through the channel 602C. Through gravity fluid at the bottom of the membrane 603C from the bottom of 601C may be delivered to the other side of the membrane 603B inside the organ plate 600B to the bottom of 604C.

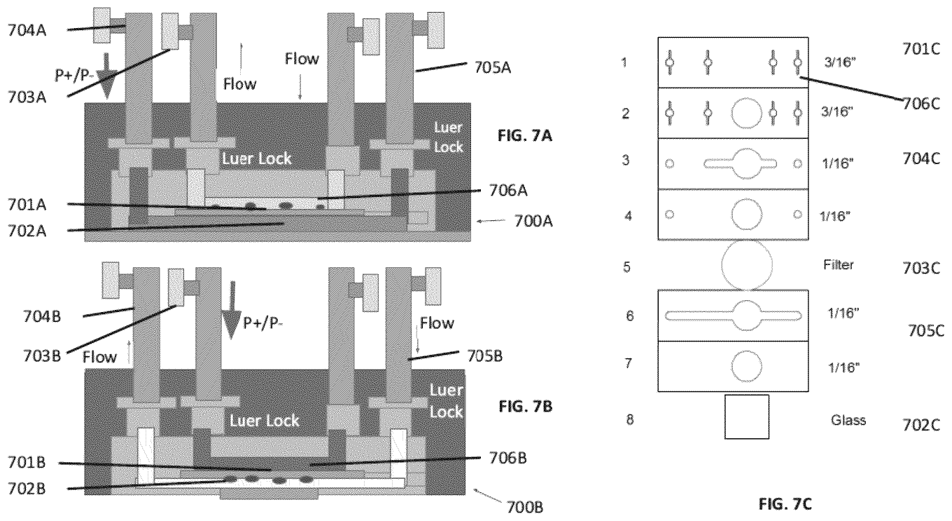

In some embodiments, at least one of co-cultured organs, cells, cell mixtures, media, and fluids may be seeded on one or both sides of the membrane 701A, 701B in the organ plate 700A, 700B and a pressurized flow to stretch the membrane 701A, 701B may be applied on the other side of the membrane 702A, 702B. In some embodiments, barrier cells and/or proteins, for example but not limited to, those found in the BBB, may prevent the at least one of the co-cultured organs, cells, cell mixtures, media, and fluids from moving across the membrane without stimulating and/or stretching the membrane. In one embodiment, FIG. 7A shows a configuration for seeding cells with fluidic flow on the membrane 701A through a top channel 706A and applying pressure pulses at the luer lock 704A that may be used for stretching the membrane 701A through the bottom channel. A second configuration for bottom channel 702A cells with fluidic flow and top channel pressure pulses at the luer lock 703B that may be used for stretching as shown in FIG. 7B. These configurations may be used for culturing muscle organs, and the configurations may need higher optical working distance for imaging the cells because the cells may be on the top of the membrane. In some embodiments, seeding cells at the bottom of the membrane may necessitate that the organ plate has to be inverted to allow gravity-based seeding and cell-adherence.

In another embodiment, to culture skin or lung cells or organs on the membrane to mimic an air/media interface, the membrane has to be porous to allow polarizing the cells and transporting media or nutrients to the cells. In order to stretch the membrane, endothelial cells may be seeded on the other side where cell medium may be present. The cells may be cultured to form a barrier that blocks gas passing through the membrane while pneumatic stretching. In order to perform fluidic perfusion, an external fluidic pump such as syringe pump may be connected to the luer lock 704A, 704B. Tall wells or tubes 705A, 705B that may be connected to the fluidic channels help remove any bubbles in the channels through gravity-release. While loading any cells in the membrane 701A, 701B there may be fluids in the top channel 706A or in the bottom channel 702B, as such the organ device may be held vertically and the fluids may be loaded from the bottom so that any bubbles within the membrane 703A, 703B may be released through a top outlet 703A and/or 704B of the organ plate 700A or 700B.

In some embodiments, the fabrication of the organ plate 700C with multiple layers of plastic chips as shown in FIG. 7C. In some embodiments, luer locks 706C may be connected to a top layer 701C. In some embodiments, a thin glass plate 702C may be attached to a bottom layer, a membrane 703C may be attached in a middle layer and top and bottom channels 704C, 705C, which may be bonded to complete the organ plate 700C.

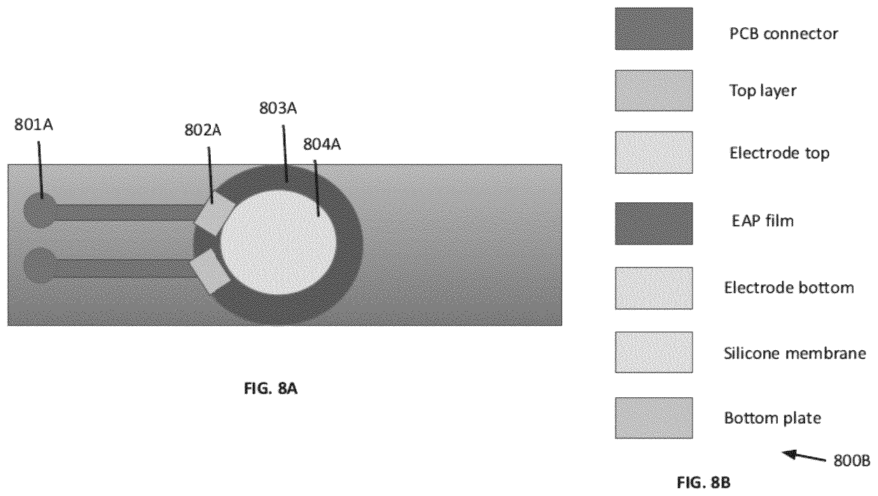

In some embodiments, electroactive polymer 803A may be bonded to a membrane 804A of an organ plate 800A that may be used for stretching by applying electrical voltage via a Printed Circuit Board (PCB) 801A as shown in FIG. 8A. In some embodiments, an electroactive polymer (EAP) with a ring shape 803A may be used to visualize the cells on the membrane. In some embodiments, the EAP may be bonded to the membrane. The contacts 802A to the electroactive polymer may be connected through a PCB 801A. In some embodiments, different layers of the organ plate 800B may be formed. One such example presented in FIG. 8B, may comprise of at least one of a PCB 801A, top and bottom fluidic channels, contact electrodes, an EAP layer, and membrane layer.

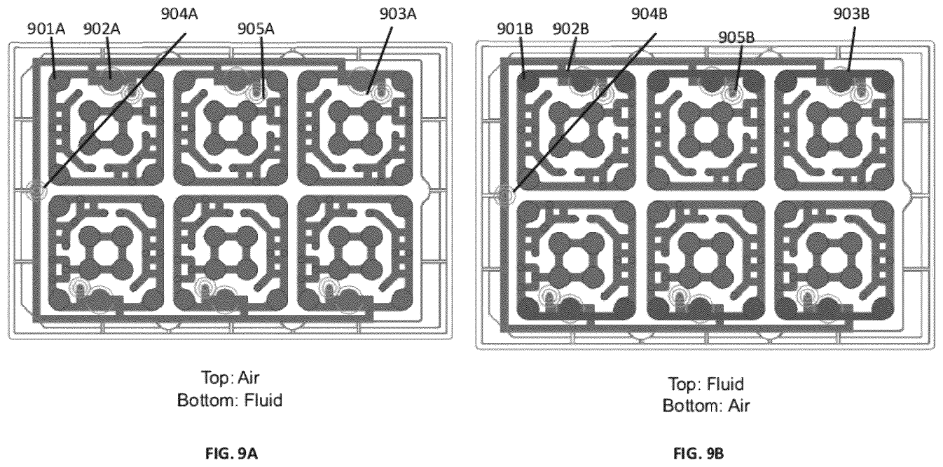

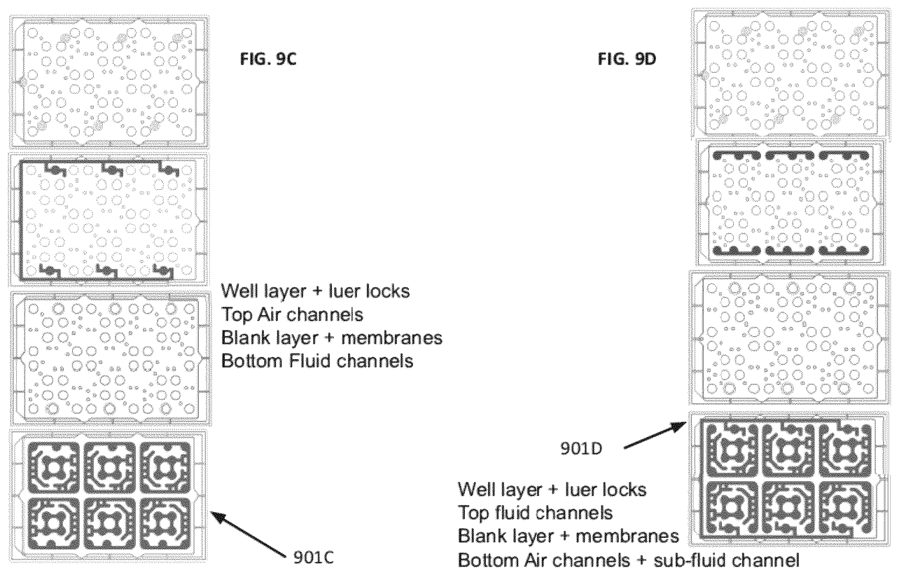

In some embodiments, stretching of organs in a high throughput organ plate may be integrated using multiple layers of organ plate. Two approaches to mimic the air/media interface may be shown as a top air 902A and a bottom fluid 901A type device, and a top fluid 901B and a bottom air 902B type device that may be implemented as shown in FIGS. 9A and 9B respectively. These two approaches may be similar to the embodiments in FIG. 7A and FIG. 7B respectively. The cells may be introduced on membranes 903A, 903B through channel inlets 905A, 905B and membranes 903A, 903B may be stretched by air pressure from a luer lock 904A, 904B. Different layers 901C of organ plate with well layer plus luer locks, top air channels, blank layers plus membranes and bottom fluidics channels may be used as shown in FIG. 9C. Different layers 901D of organ plate with well layer plus luer locks, top fluid channels, blank layers plus membranes and bottom air channels plus sub-fluid channels may be used as shown in FIG. 9D.

### 3-D Organ Culture in the Organ Plate

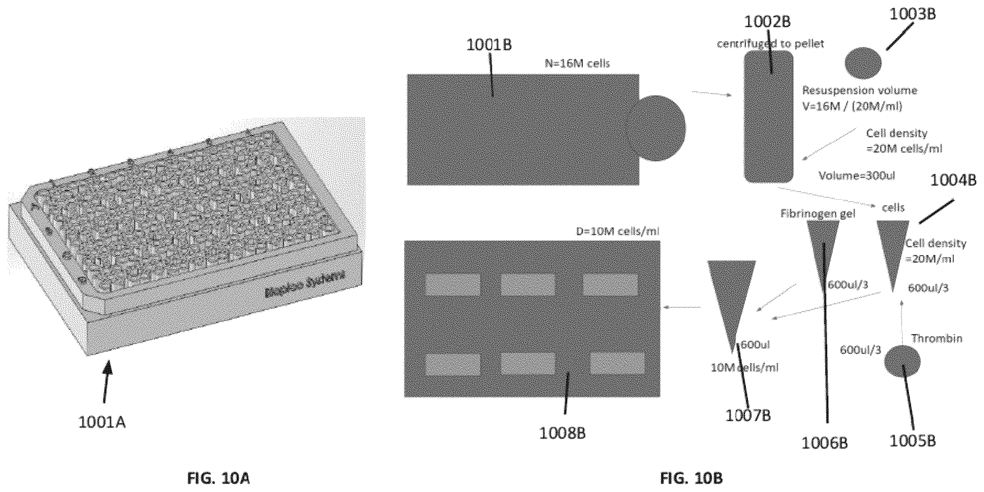

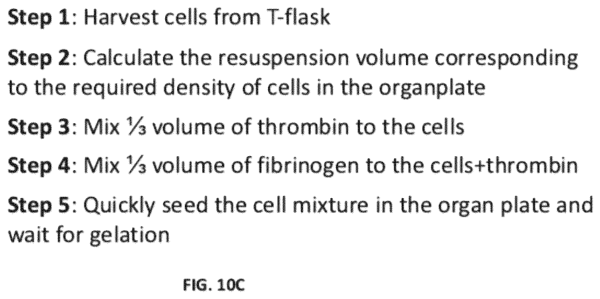

In another embodiment, an organ plate 1001A shown in FIG. 10A may have a 6×4 interacting organ system that may hold 1-2 ml of media in each organ unit. The organ plate 1001A may have two layers: a bottom layer comprising a glass-bottom channel; and, a top layer of wells. A lid with condensation rings across the wells may close the organ plate 1001A in sterile condition. Each interacting organ unit may accommodate an endothelium-epithelium co-culture of multiple organs such as liver, gut, kidney, brain, etc. The organ system has two recirculation loops and may be linked by blood-brain barrier (BBB). The endothelial cells in the outer recirculation loop may experience a unidirectional flow with programmable shear flow rates. Exchange of fluids across endothelium and epithelium may be enabled through interdigitated side channels. The organ plate may be transparent with 0.17 mm thickness at the bottom for optical imaging. In some embodiments, biochemical fluid sampling may be carried out through corner wells and epithelial-endothelial access ports. In most embodiments, the organ system may use four linear motors to perform recirculation. The linear motors may be stepper motors that may be controlled by a computer application. In some embodiments, head pressure of fluids in three position levels (low, medium and high positions) may drive gravity-driven unidirectional recirculation that mimics or stimulates recapitulating physiological fluidic shear. In some embodiments, a 20-50 microliter of a supernatant may be sampled for Liquid Chromatography/Mass Spectroscopy analysis.

For example, an organ plate may provide a multicellular environment within an extracellular matrix, a 3D structure, incorporating primary or stem cell derived cells, and immune system components with perfusion accomplished by connecting through fluidic circuits that may model the function of interconnecting tissues. The organ plate may be validated using single organs and/or coupled multi-organs such as liver, gut, kidney and brain. For example, a gut organ consists of primary intestinal epithelial cells, where as a liver organ consists of cryopreserved primary human hepatocytes, cryopreserved primary human liver sinusoidal endothelial cells, cryopreserved human Kupffer cells and human Stellate cells. Continuing with the example, the hepatocytes may be seeded at a density of 3×106 cells/mL in an epithelial channel and the other cells may be seeded in a mixture at a density range of 9×106 cells/mL, 6×106 cells/mL, and 0.3×106 cells/mL respectively.

In another example, the kidney organ may be made of primary human renal proximal tubular epithelial cells that may be seeded at 1×106 cells/mL, and human renal endothelial cells that may be seeded at 1×106 cells/mL. The intestine organ may be seeded at 0.3×106 and intestinal endothelial cells may be seeded at 0.6×106. In vitro to in vitro multi organ scaling as a ratio of cell number across the organ and in vivo to in vitro scaling for the Kilogram (human) to microgram may be performed through multiple empirical trials. In another example, a brain organ consists of IPSC derived cells of neurons, microglia, oligodendrocytes and astrocytes and blood-brain-barrier with IPSC derived endothelial cells, pericytes and astrocytes. For the BBB, endothelial cells at a concentration of 1.4×104 cells/ml may be seeded on the blood side channel and 9×105 astrocytes and 3×105 pericyte cells/ml may be seeded at the brain side. The organs may be connected to the endothelial layer in parallel and the waste from epithelial layers may be collected for analysis.

In some embodiments, 3-D organs may be cultured from the cells harvested in T-flasks 1001B mixed with fibrinogen/thrombin as shown in FIG. 10B. The cell seeding density for the organ plate may be back calculated for determining the volume of media 1003B for cell pellet 1002B resuspension. Equal volume of cells in media 1004B, thrombin 1005B and fibrinogen 1006B may be mixed together 1007B for seeding the organ plate 1008B. The steps involved in the preparation of gel and cells are shown in FIG. 10C. In some embodiments, to feed multiple cells, common media may be optimized with Minimum Essential Medium, or William's medium and iteratively supplement specific ratios by understanding the minimally essential components that may be needed for supporting organ-specific functions.

In some embodiments, the drug candidates may enter from the gut and diffuse or transport across the endothelium through the BBB to interact with the neuronal cells. Drug dosing concentrations may be determined from the unbound human highest concentration of a drug in the blood (known in the art as Cmax) for each drug. First, the expected fraction of drug unbound in media with 2% Fetal Bovine Serum may be extrapolated from plasma binding data for each drug. Dosing concentration may then be back calculated such that the unbound fraction in media may reflect relevant multiples of unbound human Cmax.

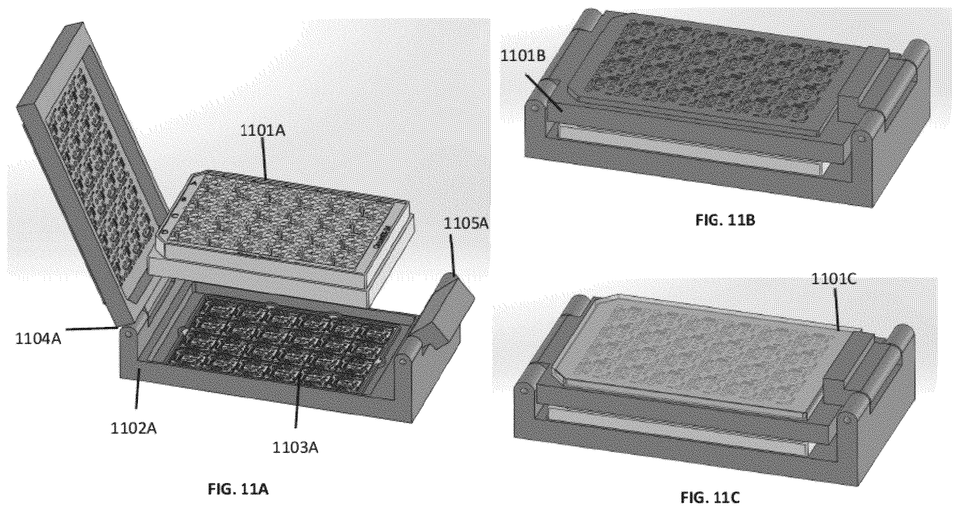

In some embodiments, after completion of organ culture experiments there may be a need to retrieve the organ samples from an organ plate. The bottom glass plate may be removed from the organ plate by thermal shock in the deep freezer. In another embodiment, the organ plate may be systematically removed from a fixture 1102A where the organ experiment was carried out. FIG. 11A shows an organ plate 1101A before closing the fixture 1102A with a plastic organ plate top 1101A, a gasket 1103A, and bottom glass is not shown (under the gasket 1103A). In some embodiments, the gasket 1103A may be above the organ plate 1101A. In most embodiments, the fixture 1102A also has a bracket 1101B that may close the organ plate with a lid (not shown). At least one gasket may be used to bond and seal at least one of the organ plate plastic and/or glass surfaces and the surfaces of the fixture 1102A by pressure fitting them with the bracket 1101B. Before seeding the cells, the organ plate may be tightly pressed as shown in FIG. 11B. The cells may be seeded and a top lid 1101C may be closed for sterile culture in an incubator. The bracket 1101B may have a hinge 1104A and a latch 1105A for fast assembly and disassembly. After the experiment, the bracket 1101A and the gasket 1103A may be removed, and the cells may be retrieved from the channels of the organ plate 1101A for post-processing.

### Manufacturing, Manipulation and Monitoring of Organs on Multiple Organ Plates

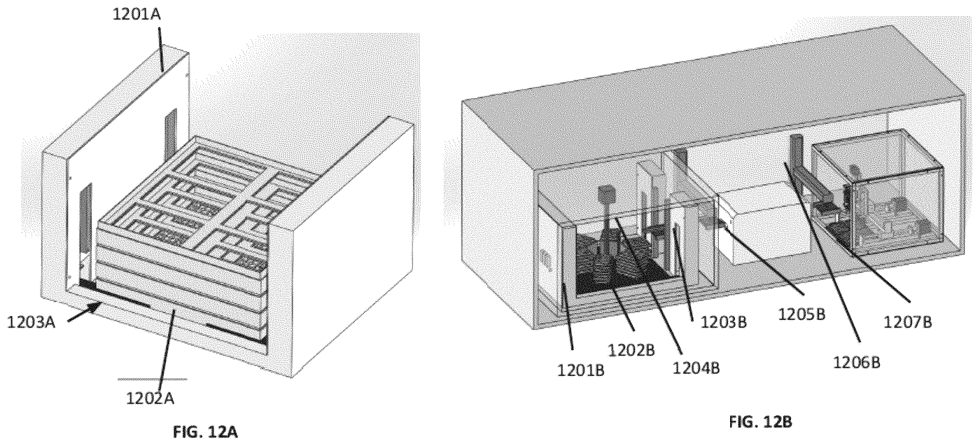

In most embodiments, multiple organ plates 1200A may be subjected to gravity-driven recirculation 1201A by stacking them in x, y, and z direction. In most embodiments, six organ plates may be stacked on a tray 1202A, and six trays may be stacked one over the other 1203A as seen in FIG. 12A. In most embodiments, to keep the trays together, a hook may be connected when stacking the plates 1200A.

In most embodiments, when tracking each organ plate 1200B in the organ system 1201B, the organ plates 1200B may be grouped in single column towers 1202B. Several such single column towers 1202B may be connected to a spinning axis 1204B, which may be used to selectively retrieve an organ plate 1200B from the organ system 1201B. In most embodiments, a Z-travel pickup or plate grabber 1203B may be operated in conjunction with the spinning axis 1204B. The entire system may be operated on a multi-axis tilt-based gravity-driven recirculation system 1210B.

In most embodiments, after retrieving an organ plate 1200B, a robotic arm 1206B may pick up the organ plate 1200B from the recirculation system 1201B may transport the organ plate 1200B to an imager 1205B. After imaging, a robotic arm 1206B may move the organ plate 1200B into a sterile laminar hood 1207B for automatic media change. After the media change, the organ plate 1200B may be put back into the recirculation system 1201B.

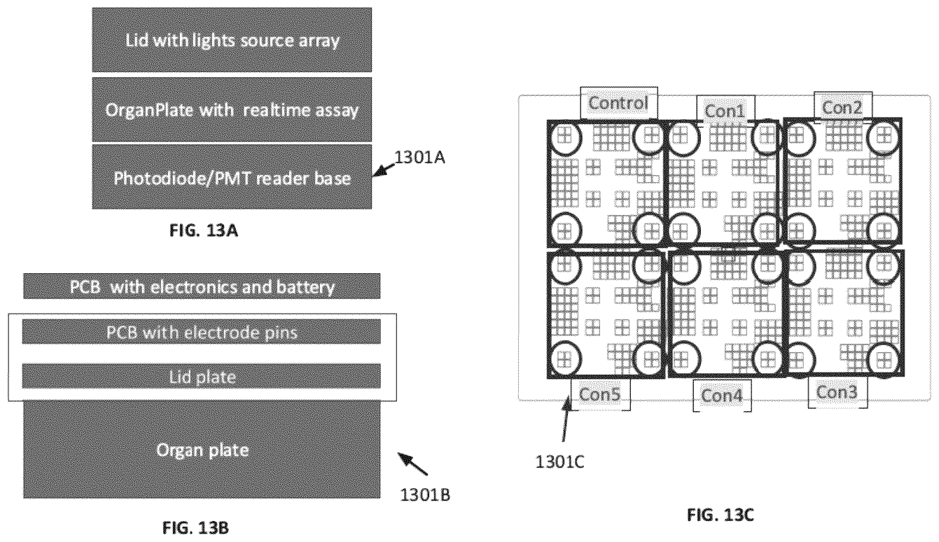

In some embodiments, the organ plate may be monitored using field potential measurements, TEER measurements, and integration with optical measurement systems. Oxygen, pH, proliferation or live/dead assays may be performed using a lid with light source array and reader base using at least one of a photodiode and Photomultiplier tube as shown in FIG. 13A. TEER measurements may be carried out using a PCB with electronics connecting to another disposable PCB with electrode pins as shown in FIG. 13B. These PCBs may be connected to the organ plate lid in a sterile fashion and may perform continuous measurement controlled by a smart device app. In some embodiments, the organ plate may be measured for multiple parameters such as chemiluminescence, fluorescence and oxygen consumption rate using a multimodal reader using a sparse array 1301C of high throughput microwell plates. FIG. 13C shows a template for 1536 microwell plates.

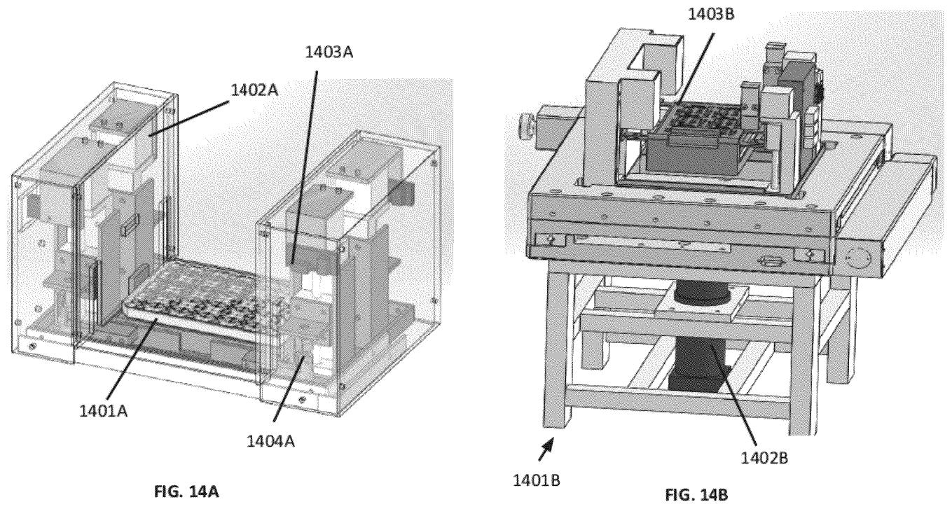

In another embodiment, an organ system for imaging an organ plate 1401A instantaneously during organ culture may be used with a built-in microscope as shown in FIG. 14A and FIG. 14B. The organ plate 1401A may be placed on a tray with an opening for imaging. The recirculation may be enabled by four or more stepper motors 1402A controlled by limit switch 1403A. The stepper motors may be connected to the tray using hooks 1404A. The microscope 1402B may hold the organ system 1401B. The optical elements for the microscope and camera 1402B may be positioned to capture the entire organ plate 1403B during recirculation. The organ plate 1403B may be tilted to provide recirculation.

### Organ Plate Assays

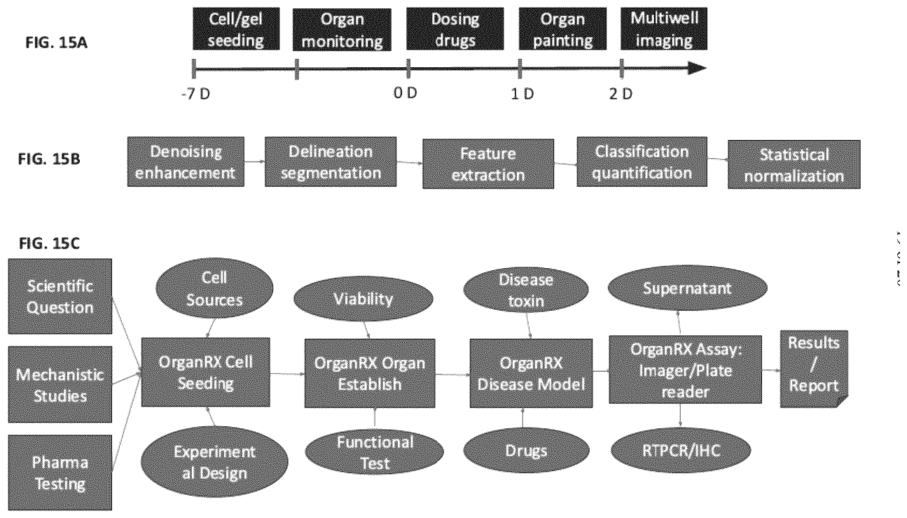

In most embodiments, the workflow for organ plate seeding and drug studies may be shown in FIG. 15A. To characterize microvascular parameters, confocal images may be analyzed using cell painting protocol. From 2D projections, percentage of area coverage of lateral vessel area (Alateral), and total branch length (Lbranch) may be computed after image processing using an artificial intelligence algorithm shown in FIG. 15B. The entire procedure for using an organ plate may be presented as in FIG. 15C. An experiment may be designed for addressing scientific questions or mechanistic studies of therapeutics or validating pharmaceutical products.

In most embodiments, organ culture may involve cell seeding in an organ plate (i.e., seeding with an organ plate trademarked as OrganRX™) for an experimental design with cell densities and cellular types. The organ model may mature for one to two weeks and may be validated using viability and functional assays. In some embodiments, a disease model of the organs may be created using at least one agent such as chemicals, high power optics, radioactivity and ultrasonic waves, etc. Pharmaceutical therapeutic agents may be used to neutralize the disease in the model. Multiple tests may be carried out from at least one non-invasive electrical and optical techniques, effluent sampling measurement using at least one technique such as immunoassay and LC/MS. Further, ends of experiment samples may be used for RTPCR (reverse transcription-polymerase chain reaction) or immunohistochemistry measurements, or both. The effluents need to be sampled from the organ plate in a sterile manner and so an automatic delivery of samples from the organ plate 1602A to a standard well plate 1604A may be designed.

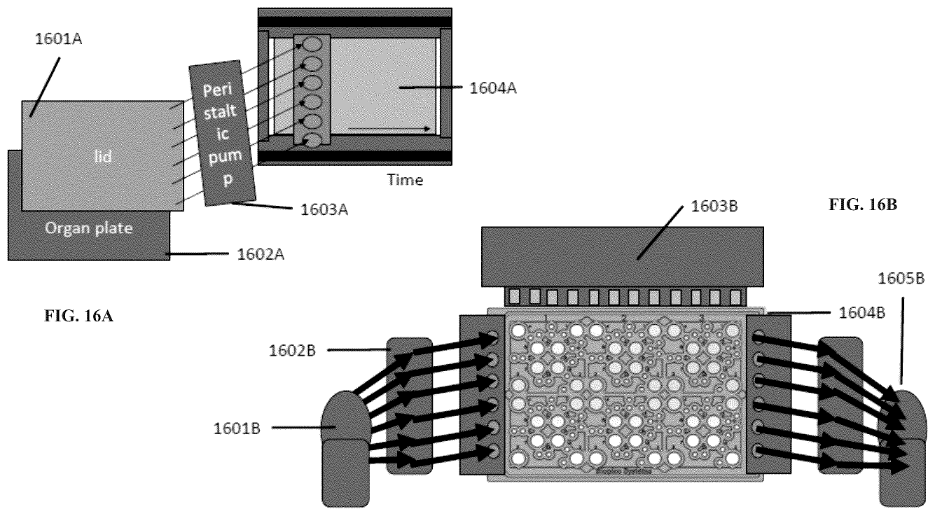

In most embodiments, as shown in FIG. 16A, an automatic method may use a microfluidic lid 1601A on the organ plate 1602A and peristaltic pumping 1603A to sample effluent media from the organ plate on standard well plates 1604A in a timely manner. In some embodiments, an automatic media exchange may be required for unattended culturing organ plates. In this embodiment, a set of peristaltic pumps 1602B with a microfluidic lid 1604B may transfer waste media 1605B out of the organ plate 1600B and may transfer fresh media 1601B into the organ plate 1600B while monitoring the organ plate 1600B using at least one sensing measurement device 1603B.

In some embodiments, various biochemical and biophysical assays may be conducted in the organ plate. The barrier function permeability may be assessed by perfusing media with small molecules of 3 kDa size. The fold change average immunofluorescence intensity may be calculated based on cell boundary length (ZO-1, occludin, and claudin-5) or vascularized area (laminin, collagen IV). In some embodiments, RNA may be extracted from different organs using TRI Reagent, and next-generation sequencing may be performed to study gene expression. After quality control and RNA-seq library preparation, the samples may undergo sequencing with a depth of approximately 50M paired-end reads per sample. The sequence reads may be trimmed to remove poor-quality nucleotides and adapter sequences. The RNA-seq data aids in the development of a mechanistic understanding of cellular signaling pathways.

### APPLICATION EXAMPLES

Multi-organ Toxicology Studies: The study of toxicity may involve the quantitative characterization of 3D neural networks using phenotypic readouts. Network bursts may be measured through field potential measurements with an adaptive threshold. Neurotransmitter and enzyme secretion may be assessed after drug treatment to study toxicity. Cytotoxicity may be evaluated by measuring LDH activity released in the media after exposure. The effects of toxic compounds on spontaneous and stimulus activity may be compared to baseline activity.

Additionally, high content image analysis methods may be used to measure concentration-dependent inhibitory effects of compounds on the complexity of neurite networks, predicting neurotoxicity. For kidney toxicity, the efficiency of proximal tubules (PT) in reabsorbing glucose may be estimated based on their reabsorption rates, PT length, and diameter. The integrity of the epithelial barrier may be investigated using fluorescently labeled dextrans. Drug dosing may be performed in the media to measure EC50/IC50 values. The organ system with access ports allows sampling of the medium and replenishment. Fluorescent inulin tracer may validate theoretical drug distribution calculations. For metabolism studies, models for absorption, first-pass metabolism, clearance, metabolite identification, induction, and transport may be used. Organ plates may enable the study of drug PK behavior in vivo, providing quantitative PBPK predictions for meaningful compound assessment. Media sampling across organs may be analyzed using HPLC/MS to measure drug PK properties. The organs may be analyzed for physiological relevance, including albumin reabsorption, alkaline phosphatase secretion, and glucose transport in the kidney; villus differentiation, mucus production, barrier function, CYP enzyme activity, and intestinal responses in the gut; and albumin production, CYP-enzyme activities, and human-specific drug toxicities in the liver. DMPK parameters with Bland-Altman analysis may be performed for individual organs and multiorgan-based experiments. Effluents from multiple compartments may be analyzed to quantify albumin, ALT, IL-6, and TNF-alpha levels. AChE activities may be measured. Predictive toxicology using in vitro data scaling to predict in vivo results may be conducted.

ADME-related applications may be carried out using model compounds, enabling predictive toxicology in regulatory decision-making. The organ system may be validated for metabolic competency and maintenance of albumin production and drug-metabolizing enzymes. Drug toxicities may be assessed by quantifying inhibition of albumin production and increases in alanine aminotransferase. Metabolites and downstream pharmacodynamics of drugs may be studied as they undergo modifications across organ systems. The brain organ assesses circulating metabolites in neuronal pharmacological activity or toxicity. Endpoints obtained from the organ plate may be integrated using physiologic parameters and mechanistic models to determine whole-body PK. Hepatotoxicity may be confirmed with immunofluorescence microscopy. Metabolic clearance guides human dose projection using ADME assays, influencing compound advancement to the clinic.

Drug Screening for Alzheimer's Disease: The prevalence of Alzheimer's disease (AD) increases significantly with age, affecting 0.4-1% of individuals aged between 60-65 years and 15-33% of those over 90 years old. Despite numerous attempts to develop disease-modifying AD drugs, including monoclonal antibodies, secretase inhibitors, and tau aggregation inhibitors, many have failed. Moreover, transgenic mice studies have not been reliable in predicting potential therapeutics for AD. To better understand AD pathophysiology, studying the presence of AD genetic mutations with isogenic controls may be crucial. This approach may reveal ion channel dysfunction, dystrophic neurites, and synaptic damage in the presence of elevated AP levels in the human AD brain. By comparing results across genetic variants, cell types, and analysis modalities, convergent molecular mechanisms of the disease may be identified. Organoid cultures have been used in such studies, but they lack complete structural organization, cellular diversity, vascularity, and immune components, limiting their physiological relevance.

To address this, the above invention aims to create brain organ models using isogenic cell lines to develop new therapeutics in a target-specific manner. These models may incorporate scalable, 3D brain-tissue organization coupled with blood-brain barrier (BBB) perfusion to simulate multiple aspects of AD pathophysiology. By mimicking blood flow and inducing disease conditions in combination with cellular barriers, this system may allow the modeling of secondary target organs and improves organ viability, differentiation, and polarity. The system may shed light on the major genetic risk factor for AD (ApoE4) and may provide valuable insights for future AD therapeutics based on ApoE4. This multicellular organ culture system may enhance cross-talk between 3-D neural networks involved in AD and vascularized BBB, advancing the targeting of neuron and glia-mediated Aβ clearance and lipid biogenesis/metabolism. These organ models may be significant in studying phenotypes under endogenous and more physiological levels of mutant protein expression. While some drugs have entered clinical trials, further evidence may be needed to determine appropriate doses and toxicities using clinically relevant organ models, facilitating discussions with the FDA. This comprehensive approach aims to revolutionize AD research and potentially lead to effective treatments for this devastating neurodegenerative disease.

Diabetes: Pancreatic islet transplantation offers significant improvements in glycemic control and prevention of life-threatening severe hypoglycemia for patients with type 1 diabetes (T1D). However, the intricate pathophysiology of T1D, characterized by tightly controlled microenvironments and specific immune cells, poses challenges for successful grafting. Despite advancements in understanding the genetic, molecular, and cellular mechanisms of T1D, clinical treatments still face hurdles without an integrative approach to observe the autoimmune diabetes pathophysiology. While animal models have provided insights into these integrative processes, enhancing the relevance of discoveries for human patients requires the utilization of human cells in the tissue and immune environment.

Bioengineered therapeutic strategies using organ systems may enable accurate modeling and monitoring of the entire spectrum of T1D pathophysiology, spanning from disease initiation to manifestation and beyond. Early stages of T1D disease progression may be mimicked by introducing activated immune cells to migrate into the islets and observing their effects. The endothelial cells may form a two-way flow path in the vascular vessel network, enabling precise supply and exit of the islets with essential nutrients, oxygen, and physiological molecules like glucose, hormones, and metabolites. Through cross-talk between vascularized islets, immune cells, antibodies/antigens, and effector molecules, surveillance may occur across integrated organs and multi-organs, effectively bridging the gap between in vitro systems and clinical treatments. For instance, insulin secretion promoting glucose uptake by the liver organ may be reflected in the glucose concentration, demonstrating a feedback loop between the liver and islets. Additionally, the use of patients-matched peripheral blood mononuclear cells (PBMC) compared to healthy volunteers may demonstrate the feasibility of using these cells to model relevant cell-cell interactions and interpret T1D pathogenesis. In summary, the development of therapeutic strategies using organ system as described above may provide a comprehensive understanding of T1D pathophysiology, facilitating targeted treatments and potentially improving outcomes for patients dealing with this complex autoimmune disease.

Gut-Liver-Kidney Multiorgan Model for Nephrotoxicity: Physiological processes involving glomerular filtration, tubular secretion, and tubular reabsorption present challenges for animal models to fully replicate the diverse causes, mechanisms, and manifestations of kidney diseases in humans. Nephrotoxicity, while only accounting for 2% of drug development failures in preclinical studies, increases significantly to tenfold during Phase 3 and clinical settings, leading to acute kidney injury. Current preclinical cell culture and animal models lack reliability in predicting adverse drug effects on the kidney. To address this issue, there may be a growing need to develop better in vitro approaches to mimic kidney function.

Combining gut-liver-kidney organs may offer a promising solution for evaluating compounds and understanding mechanisms of action, leading to more effective therapeutic solutions while reducing clinical and commercial failures. High throughput gravity-driven organ recirculation systems, with fluid shear stress, may enhance the functionality of developed kidney organs, resulting in improved albumin transport, glucose reabsorption, cisplatin toxicity, P-glycoprotein (Pgp) efflux transporter activity, and brush border membrane alkaline phosphatase activity. The inclusion of extracellular matrix (ECM) coating and unidirectional apical flow may lead to the formation of mature intestinal tubule phenotypes with villus-like structures. By incorporating various cell-cell interactions and factors affecting kidney tubules, such as fluid flow and cellular metabolic functions, the system may better mimic the complex organ-level responses, including barrier function and cellular interfaces. The GLK system, when integrated with fluidic perfusion in a portable format, may be kept for several 30-day periods in an incubator, enabling continuous organ interaction and adaptability for optical imaging. This system allows for the reconstitution and visualization of organ-level responses and offers a viable screening strategy for accurately detecting nephrotoxicity early in drug development. In summary, the integration of gut-liver-kidney organs and the GLK system, with fluid shear stress and other factors, may present a promising approach for studying kidney function, drug efficacy, and potential toxicity. This advancement may lead to improved drug safety and reduced costs of drug development by identifying potential kidney-related issues earlier in the process.

Environmental Toxicology: The etiology of neurological diseases explores the role of intestinal microbiota and xenobiotics in the gut-brain axis, influencing treatment options. Genetic variations in xenobiotic-mediated neurotoxic signaling may lead to inflammation, oxidative stress, and neuronal dysfunction. The reciprocal influence between the liver and gut affects biosynthesis pathways and the integrity of the intestinal epithelial barrier, impacting the delivery of neurotoxins or metabolites to neurodegenerative sites. Understanding these multi-organ communications and genetic diversity may be vital across multiple organ models. Genotype×environment interactions (GxE) between genetic risk factors and environmental exposures may disrupt learning, memory, and contribute to neurodegenerative diseases. However, limited information supports this GxE hypothesis.

Developing interconnected multi-organ 3D tissues for fundamental toxicity studies with xenobiotic metabolites may provide valuable insights. A gravity-driven recirculation system with fluid shear stress may enhance gut-brain axis cross-talk and vascularization across the blood-brain barrier, enabling continuous toxicity studies. Incorporating isogenic IPSC lines ensures reproducibility in toxicity assays among researchers and between healthy and pathological cells. Utilizing pharmaceutical toxicity assays in well-characterized disease mutations in organ plates may predict drug efficacy and safety, considering individual patient variations due to genetic and environmental factors, thus avoiding wastage of resources and time. Integrating local immune cells and the commensal microbiome into the organ system may accelerate the discovery of personalized precision therapeutics based on drugs and microbiome interactions. The organ system provides in-vivo recapitulations for population-level genetic variability in dose-response relationships, may aid in understanding and addressing neurological disease complexity.

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice. Further, like reference numbers and designations in the various drawings indicated like elements.

Whereas many alterations and modifications of the present invention will no doubt become apparent to a person of ordinary skill in the art after having read the foregoing description, it is to be understood that the particular embodiments shown and described by way of illustration are in no way intended to be considered limiting.

It is noted that the foregoing examples have been provided merely for the purpose of explanation and are in no way to be construed as limiting of the present invention. While the present invention has been described with reference to exemplary embodiments, it is understood that the words, which have been used herein, are words of description and illustration, rather than words of limitation. Changes may be made, within the purview of the appended claims, as presently stated and as amended, without departing from the scope and spirit of the present invention in its aspects. Although the present invention has been described herein with reference to particular means, materials and embodiments, the present invention is not intended to be limited to the particulars disclosed herein; rather, the present invention extends to all functionally equivalent structures, methods and uses, such as are within the scope of the appended claims.

1. An organ plate device comprises:
at least three wells, whereas a volume of media is placed in at least one of the at least three wells;
multiple loops in at least one of series and parallel fluidic paths; whereas the at least three wells are connected to the fluidic paths;
endothelial and epithelial regions connected by bridge channels; whereas the bridge channels are connected to the fluidic paths;
at least one fluid delivered into at least one port of the epithelial region; whereas the at least one fluid stops at an expansion channel after the bridge channels; whereas the at least one fluid includes at least one of a gel, liquid, cells, and cell mixtures;
whereas a co-culture of at least one modeled organ is cultured in at least one of the bridge channels.

2. The organ plate device in claim 1, further comprising a plurality of micropillars; whereas that the plurality of micropillars separate the at least one fluid; whereas the plurality of micropillars connect at least two of the parallel fluidic paths together.

3. The organ plate device in claim 1, whereas the bridge channel stops the at least one fluid at the expansion channel area.

4. The organ plate device in claim 3, whereas the endothelial regions and epithelial regions fluids are stopped at the bridge channels with a hydrophobic coating on at least one of a glass layer and any part of the bridge channels.

5. The organ plate device in claim 1, whereas the co-culture of at least one modeled organ have cells comprising at least one of epithelial cells, endothelial cell, stromal cells, immune cells relevant to the at least one modeled organ; whereas the at least one modeled organ includes brain organs, kidney organs, lung organs, immune system organs, liver organs, gastrointestinal organs, heart organs, skin organs, and muscle organs.

6. The organ plate device in claim 5, whereas at least one of the volume of media and the one fluid contain immune cells that interact with the co-culture of the at least one modeled organ.

7. The organ plate device in claim 1, where a transwell insert is placed in at least one of the at least three wells.

8. The organ plate device in claim 1, further comprising a fluidic lid; whereas at least one of wells of the organ plate device is connected using the fluidic lid to a second well of the organ plate device that are not connected otherwise.

9. The organ plate device in claim 8, further comprising:
external reservoirs;
a mixing reservoir; and
a second reservoir, whereas the second reservoir is used for waste media;
whereas at least one of the external reservoirs, the mixing reservoir, and the second reservoir are connected to the organ plate device through the fluidic lid.

10. The organ plate device in claim 1, further comprising:
at least one of a pneumatic pressure pump and vacuum;
a stretching membrane, whereas the co-culture of the at least one modeled organ is placed on at least one of a first side and a second side of the stretching membrane;
whereas the at least one of a pneumatic pressure pump and vacuum is connected to the organ plate device; whereas the at least one of a pneumatic pressure pump and vacuum is used to stretch the stretching membrane.

11. The organ plate device in claim 10, whereas the stretching membrane is stretched by a ring electro-active polymer membrane bonded to the stretching membrane.

12. The organ plate device in claim 10, whereas the co-culture of the at least one modeled organ is placed on the first side of the stretching membrane; whereas the media is held on the second side of the stretching membrane by at least one of barrier cells and proteins; whereas when the at least one of a pneumatic pressure pump and vacuum apply pressure to the least one of the first side and the second side of the stretching membrane causes the co-cultured of the at least one organ to mature as a result of the stretching membrane being stretched.

13. The organ plate device in claim 12, whereas the co-culture of the at least one organ on the stretching membrane mimics at least one of muscle organs, lung organs, and skin organs.

14. The organ plate device in claim 1, whereas the media is recirculated using gravity driven flow by tilting corners of the organ plate device to form unidirectional flow recapitulating physiological fluidic shear.

15. The organ plate device in claim 14, whereas the volume of media is recirculated across a plurality of organ plate devices connected in at least one of series and parallel using gravity driven flow.

16. The organ plate device in claim 1, further comprising a fixture; whereas the organ plate device is placed in the fixture and is sealed using a latch and a bracket on a hinge that presses against a gasket to create a sterile closed system.

17. The organ plate device in claim 1, further comprising:
a lid;
measurement sensors;
whereas the measurement sensors are placed in the lid to monitor characteristics of the co-culture of the at least one organ cell.

18. The organ plate device in claim 17, whereas the characteristics of the co-culture comprises at least one of chemiluminescence, fluorescence, and oxygen consumption.

19. The organ plate device in claim 17, whereas the measurements sensors comprises field potential sensors, TEER sensors, optical sensors, oxygen sensors, and pH sensors.

20. An organ plate system, comprising:
at least one organ plate;
a robotic arm;
a recirculation device;
a spinning platform;
a moving plate grabber;
an imaging device;
an automatic media change dispenser;
whereas the at least one organ plate in the recirculation device is moved by a robotic arm to the imaging device and the automatic media change dispenser before being moved back to the recirculation device;
whereas the at least one organ plate comprises:
at least three wells, whereas a volume of media is placed in at least one of the at least three wells;
multiple loops in at least one of series and parallel fluidic paths; whereas the at least three wells are connected to the fluidic paths;
endothelial and epithelial regions connected by bridge channels; whereas the bridge channels are connected to the fluidic paths;
at least one fluid delivered into at least one port of the epithelial region; whereas the at least one fluid stops at an expansion channel after the bridge channels; whereas the at least one fluid includes at least one of a gel, liquid, cells, and cell mixtures;
whereas a co-culture of at least one modeled organ is cultured in at least one of the bridge channels; and
whereas the imaging device monitors the co-culture of the at least one of the modeled organ.
